(19) United States
(12) Patent Application Publication
(10) Pub. No.: US 2022/0125622 A1
(43) Pub. Date: Apr. 28, 2022

(54) CONTROLLED-RELEASE DEVICE

(71) Applicant: POLITECNICO DI MILANO, Milano (IT)

(72) Inventors: Marco FERRONI, Milano (IT); Francesco DE GAETANO, Milano (IT); Matteo Giuseppe CEREDA, Milano (IT); Federica BOSCHETTI, Milano (IT)

(73) Assignee: POLITECNICO DI MILANO, Milano (IT)

(21) Appl. No.: 17/436,201

(22) PCT Filed: Mar. 11, 2020
(86) PCT No.: PCT/IB2020/052153

§ 371 Date: Sep. 3, 2021

(30) Foreign Application Priority Data
Mar. 12, 2019 (IT) 102019000003597

(51) Int. Cl. A61F 6/14; B65B 1/04; B65B 3/00; B65B 7/28

(52) U.S. Cl. CPC A61F 6/148; B65B 7/28; B65B 3/003; B65B 1/04

(57) ABSTRACT

An implantable and/or injectable device made of biodegradable material which comprises two hollow volumes where said device comprises a cylinder, which comprises a top portion and a bottom portion, where the top end and bottom end of said cylinder are open and via said openings on said top and bottom ends a first hollow volume and a second hollow volume are respectively accessed, where said first hollow volume and second hollow volume are separated from each other by a partition and are characterized in that the side wall of said first hollow volume has a thickness which is smaller than the thickness of the side wall of said second hollow volume. A method for manufacturing, filling with at least one active agent and closing said device and the use of the said device filled with at least one active agent for the treatment and/or the prevention of pathologies which require repeated and programmed administration over time.

The present invention relates to a biodegradable device which can be injected and/or implanted in the human or animal body and which comprises at least one active agent. The device allows a programmed release of the active agent, where “programmed release” is understood as meaning a release of predefined doses of said at least one active agent, namely a release of quantified amounts, at separate predefined moments in time.

The present invention relates furthermore to a method for manufacturing, filling and closing said device.

Finally, the use of said device for the treatment of pathologies which require the repeated and programmed administration of at least one active agent is claimed.

### PRIOR ART

A series of pathologies which require repeated programmed treatment, namely treatment at times and in doses which are repeated and predefined, are known.

By way of example, the preferred treatment for maculopathy, the most common form of which is age-related macular degeneration (AMD), consists in intravitreal injections of drugs based on agents which inhibit growth factors of the endothelium (anti-VEGF drugs). An essential condition for the effectiveness of the treatment is that the tissue should be exposed periodically to the anti-VEGF drug.

Treatments with repeated injections pose compliance difficulties for the patient as well as representing a considerable cost for the health care service.

WO2011097634 describes an ocular implant for the release at two separate times of two drug doses, where the implant is activated by a laser so as to break and allow the release at the predefined time.

The implant described in WO2009097468 also controls the release from the outside; activation of the implant is in fact obtained with a light source.

US2005244465 describes an ocular implant which has a sandwich structure, where the central portion comprises the drug and the two outer layers are made of polymeric material. The form parameters of said sandwich structure, together with the properties of the polymeric material, adjust the release of the drug over time. The solution illustrated has the drawback that a biodegradable polymer material is used for manufacture, which results in the release of undesirable products which cause deterioration of the eye.

The present invention relates to a programmed-release device which solves the constructional design problems which the devices according to the present state of the art are unable to solve.

### DESCRIPTION OF THE FIGURES

FIG. 1 shows, in views A to F, the steps for assembly of an embodiment of an implantable and/or injectable device according to the present invention which is a cylinder: (A) providing the open, innermost, hollow body and filling it; (B) closing the innermost hollow body; (C) providing a further open hollow body and filling it; (D) inserting the innermost hollow body inside the further hollow body; (E) providing the outermost hollow body and filling it; (F) inserting the further hollow body inside the outermost hollow body.

FIG. 2 shows a vertical cross-section through an embodiment of an implantable and/or injectable device according to the present invention.

FIG. 3 shows a vertical cross-section through a hollow body included in a further embodiment of the device according to the present invention, with a conical engaging closure.

FIG. 4, in views A and B, shows a further embodiment of the closure of the device according to the present invention.

FIG. 5 shows, in views A to F, the steps of assembly of an embodiment of an implantable and/or injectable device according to the present invention: (A) providing the open innermost hollow body and filling it; (B) closing the innermost hollow body; (C) providing a further open hollow body and filling it; (D) inserting the innermost hollow body inside the further hollow body; (E) providing the outermost hollow body and filling it; (F) inserting the further hollow body inside the outermost hollow body.

FIG. 6 shows a further embodiment of the device according to the present invention: (A) vertical cross-section; (B) perspective view; (C) view from above, front elevation view, and view from below.

### DESCRIPTION OF THE INVENTION

The present invention relates to an implantable and/or injectable device which comprises at least two hollow bodies which are inserted one inside the other, where said at least two hollow bodies are made of biodegradable material. Said at least two hollow bodies delimit: i) a volume, called “core”, inside the innermost of said hollow bodies, and ii) one or more volumes, called “annular space”, situated between said innermost hollow body and the hollow body on the outside thereof. Said core and said at least one annular space are designed to contain doses of an active agent. Said hollow bodies comprise cylinders.

In a preferred embodiment, the surfaces of said hollow bodies are impermeable continuous surfaces which become permeable depending on the post-implantation and/or injection time of said device. The side surface, or wall, of each of said cylinders is an impermeable continuous surface and, preferably, at least one or both of the bases of said cylinders are also impermeable continuous surfaces which become permeable depending on the post-implantation and/or injection time of said device.

The number of hollow bodies inserted one inside the other and forming the implantable device determines the therapeutic regimen. For example, an implantable device formed by two hollow bodies inserted one inside the other, and therefore by two biodegradable layers, is able, after the implant, to perform the release of two doses of an active agent. An implantable device formed by three hollow bodies inserted one inside the other is able, after the implant, to perform the release of three doses of an active agent. During injection and/or implantation of said device, it is possible to administer a further dose of said active agent, where said further dose is simultaneously administered during injection and/or implantation of said device and is not contained therein. Embodiments which comprise up to twenty hollow bodies inserted one inside the other, preferably up to 10, or up to five, and preferably three hollow bodies inserted one inside the other, are envisaged.

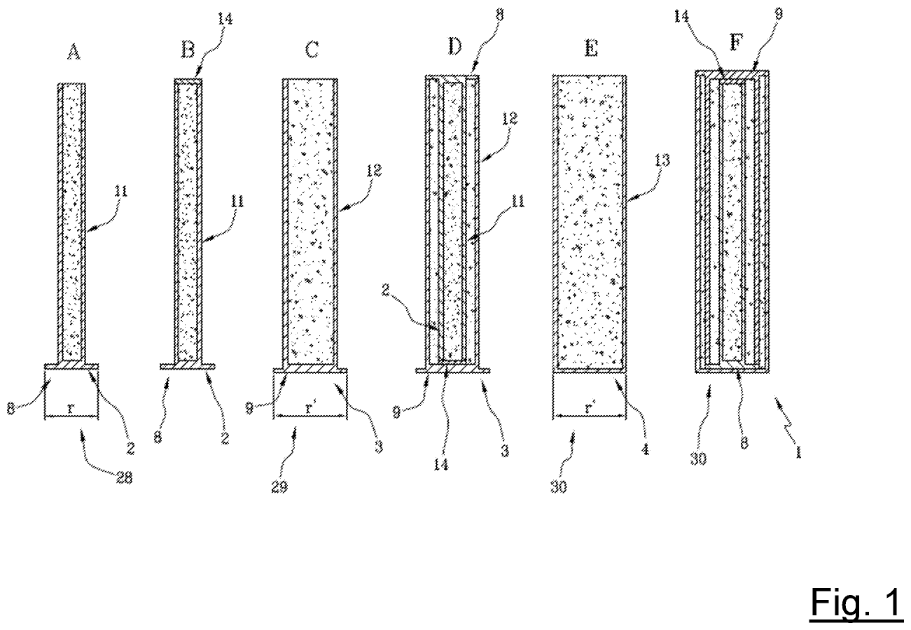

With reference to FIGS. 1, 2 and 5, purely by way of a non-limiting example, a device 1 which comprises three hollow bodies inserted one inside the other is described here in detail.

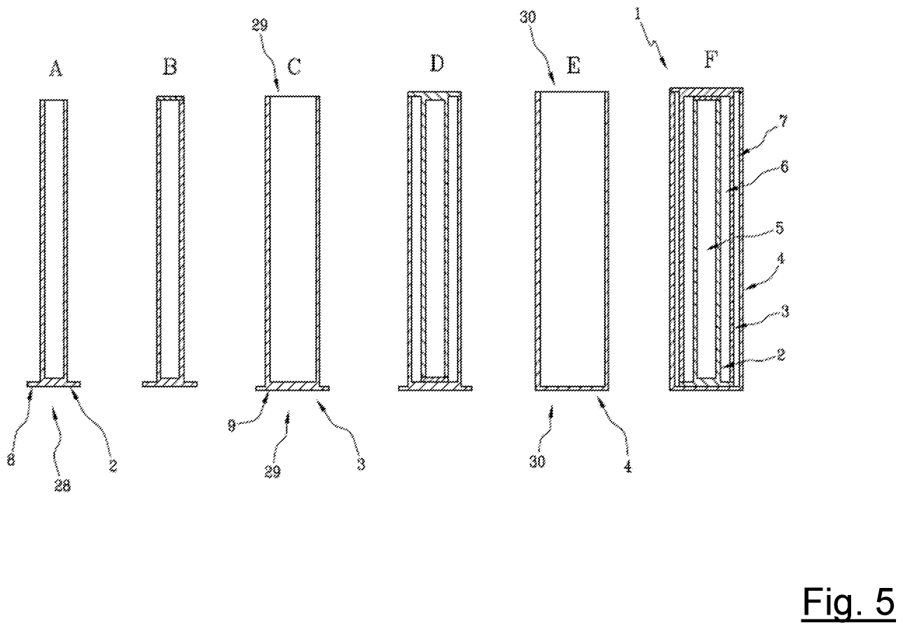

FIGS. 1, 2 and 5F show a vertical cross-section through said device 1 and show an innermost hollow body 2 inserted inside an intermediate hollow body 3 in turn inserted inside an outer hollow body 4. Said inner hollow body delimits a core 5, said intermediate hollow body 3 delimits an annular space 6, and said outer hollow body 4 delimits an annular space 7. Said hollow bodies 2, 3 and 4 comprise for example cylinders with a circular or elliptical base or a regular or irregular polygonal base, for example the base is a triangle, a square or a pentagon; in a preferred embodiment it is cylinder with a circular base. For the purpose of the present description, each of said hollow bodies is defined as being a cylinder 2, 3, 4 which may be prism with a triangular, square, pentagonal, n-gonal, elliptical or circular base, which comprises at one of the bases a base support 8, 9, with reference to FIGS. 1 and 5, which may be triangular, square, n-gonal, elliptical or circular. Said base supports 8, 9 have an area A, A′, where said areas A, A′ of said base supports 8, 9 are the same as or greater than the areas of the base 29, of the cylinder inside which said hollow body will be inserted, and can be inserted inside any further hollow body inside which said hollow body will be inserted or, with reference to the embodiment shown in FIG. 5, said area A of said base support 8 is the same as or greater than the area of the base 29 of the cylinder which forms said hollow body 3, such that said base support 8 acts as a lid for the hollow body 3 once inserted in it, as shown in FIG. 5D, being insertable within the cylinder which forms said hollow body 4. The area A, A′ of said base supports 8, 9 is greater than the base area 28, 29 of the cylinder 2, 3 with which said base support 8, 9 is associated, such that said base support 8,9 projects from the cylinder with which it is associated, as shown in the views 5B and 5C, and is the same as or greater than the area of the base 29, 30 of the cylinders 3 and 4 inside which said cylinders 2, 3 are inserted, respectively. In other words the area A of the base support 8 of the cylinder 2 is greater than the base area 28 of the said cylinder 2 and is the same as or greater than the area of the base 29 of the cylinder 3 such that said base support 8 may act as a lid once it has been inserted inside said cylinder 3, said area A being insertable within the further cylinder 4. With reference to the embodiment shown in FIG. 5A, said innermost hollow body 2 has a base support 8 with an area A. Said intermediate hollow body 3 (see FIG. 5C) is a cylinder which has a base with area A and a base support 9 with area A′. Said outermost hollow body 4 (see FIG. 5E) is a cylinder which has a base area A′.

In a preferred embodiment, said base supports 8, 9 are circular.

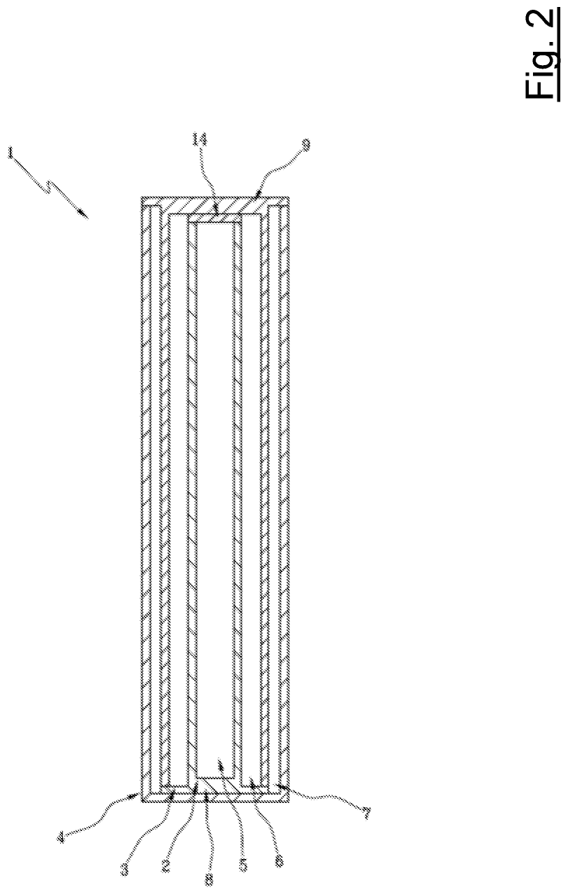

In an even more preferred embodiment, with reference to FIG. 1, said hollow bodies 2, 3 and 4 are cylinders with a circular base. In this embodiment, each of said hollow bodies 2, 3 which form said implantable/injectable device, except for the outermost hollow body 4, characteristically consists of a cylinder having a circular base support 8, 9 with radius r, r′, where the lengths of said radii r, r′ of said base supports 8, 9 are the same as or greater than the lengths of the base radius of the cylinder inside which said hollow body will be inserted, and smaller than the length of the base radius of any further hollow body inside which said hollow body will be inserted, in other words, with reference to the embodiment shown in FIG. 1, said radius r of said base support 8 has a length which is the same or greater than the length of the base radius of the cylinder which forms said hollow body 3 and smaller than the length of base radius of the cylinder which forms said hollow body 4. The length of said radii r, r′ of said base supports 8, 9 is greater than the base radius of the cylinder 2, 3 with which said base support is associated and smaller than the base radius of the cylinder 3 and 4 inside which said cylinders 2, 3 are inserted, respectively. With reference to the embodiment shown in FIG. 2, said innermost hollow body 2 has a base support 8 with radius r. Said intermediate hollow body 3 is a cylinder which has a base radius with length r and a base support 9 with radius r′. Said outermost hollow body 4 is a cylinder which has a base radius with length r′.

In said device the conditions as regards the dimensions of the base supports and the base area are replicated for each further hollow body forming part thereof.

The geometrical form of the device according to the present invention advantageously solves the technical problem of assembling and filling a device comprising bodies inserted inside each other, having dimensions which are suitable for an implantable and/or injectable device. The advantages arising from said structure will be evident from the process which results in assembly of the implantable/injectable device.

Said hollow bodies 2, 3 and 4 are made of biodegradable material which is preferably magnesium or a magnesium alloy. Magnesium alloys such as aluminium, lithium, calcium, zinc or manganese alloys form particularly preferred embodiments.

In a preferred embodiment, said device is made of magnesium alloy JDBM, an alloy of Mg-2.5Nd-0.2Zn-0.4Zr (wt %, JDBM) which has good mechanical and corrosion-resistance properties. Alternatively, said alloy is JBDM-2, Mg-2.2Nd-0.1Zn-0.4Zr (wt %, indicated as JDMB-2).

The advantages which can be obtained with the use of compounds of magnesium in an implantable and/or injectable device are associated with the complete reabsorption, owing to the limited corrosion-resistance in damp environments, and a high biocompatibility of the corrosion products.

The thickness of the magnesium layers which form said hollow bodies determines the degree of degradation thereof, where a greater thickness corresponds to a longer degradation time.

The device according to the present invention is impermeable and becomes permeable depending on the post-implantation and/or injection time of said device. In a preferred embodiment, said permeability is obtained with the dissolution of the wall of the outermost hollow body. Said dissolution, which occurs depending on the post-implantation and injection time and the thickness thereof, results in release of the active agent contained in the outermost hollow body. Thereafter, and following exposure of the wall of the inserted hollow body to the implantation and/or injection environment, degradation of the wall of the hollow body inserted inside outermost hollow body occurs, with release of the amount of active agent contained therein.

In one embodiment, the thickness of the wall of said hollow bodies is the same for each of said hollow bodies. In an alternative embodiment, advantageously applied when said active agent is in liquid form, the thickness of the wall of said hollow bodies varies, increasing, in the direction from said outermost hollow body to said innermost hollow body.

In a preferred embodiment, the degradation time of each hollow body is 2 weeks, or 3 weeks, or 4 weeks, or 6 weeks, or 8 weeks, or 10 weeks or 12 weeks.

Said core 5 and said one or more annular spaces 6, 7 in one embodiment have the same volume as each other. In an alternative embodiment, they have volumes different from each other.

In one embodiment, the implantable and/or injectable device comprises a cylinder and has a height of about 10 mm or about 5 mm and a diameter of the base, where circular, or of the circle inside which said base can be inscribed, equal to about 0.8 mm, 0.7 mm or 0.6 mm, for ophthalmic applications. Said cylinder has a height of up to 40 mm and a diameter of the base, where circular, or of the circle inside which said base can be inscribed, of up to 4 mm in the case where said device is intended for applications other than ophthalmic applications.

The present invention relates furthermore to a method for manufacturing, filling and closing and closing an implantable and/or injectable device for the controlled release of an active agent.

Said method, with reference to FIG. 1, comprises the following steps:
a) Providing single, open, hollow bodies 2, 3, 4, each hollow body 2, 3, 4 being cylindrical and comprising the side walls 11, 12, 13 and the bases 28, 29, 30, characterized in that each of said open hollow bodies which will form the inserted hollow bodies 2, 3 has, on said base 28, a base support 8 and, on said base 29, a base support 9 with an area A, A′, where the areas of said base supports 8, 9 are the same as or greater than the areas of the base of the cylinder 3, 4 inside which said hollow body 2, 3 will be inserted and smaller than the area of the base of the further cylinder 4 inside which said hollow body 3 will be inserted. In other words, the area A of the base support 8 is greater than the area of the base 28 of the cylinder with which said base is associated; the outermost hollow body 4 has a base 10 and does not require a base support;
b) Filling with at least one active agent the innermost internal volume of said hollow bodies, called core 5;
c) Closing said innermost hollow body 2 with a lid which is the top end 14 of said cylinder which forms said innermost hollow body 2;
d) Filling the hollow body 3 with at least one active agent;
e) Inserting inside said hollow body 3 said innermost hollow body 2, where the top end 14 of said innermost hollow body 2 rests on the base 29 of said hollow body 3 and said base support 8 of said hollow body 2 forms the top end of said hollow body 3;
f) Filling the hollow body 4 with at least one active agent;
g) Inserting into said hollow body 4 said hollow body 3 inside which said innermost hollow body 2 is inserted, where the base support 9 of said hollow body 3 forms the top end of said hollow body 4;
h) Repeating said steps f and g for each further hollow body included in said device.

In a preferred embodiment, said base supports have a circular form.

In an even more preferred embodiment, said hollow bodies are cylinders with a circular base. In particular, where said hollow bodies are cylinders and base supports are circular, said method comprises:
a) Providing single, open, hollow bodies 2, 3, 4, each hollow body 2, 3, 4 having a cylinder form and comprising the side walls 11, 12, 13 and a bottom end 28, 29, 30, characterized in that each of said open hollow bodies which will form the inserted hollow bodies 2, 3 has a base support 8, 9 with radius r, r′, where the length of said radii r, r′ of said base supports 8, 9 have a length the same as or greater than the base radius of the cylinder 3, 4 inside which said hollow body 2, 3 will be inserted and smaller than the base radius of the further cylinder 4 inside which said hollow body 3 will be inserted. In other words r is greater than the base radius of the cylinder with which said base is associated. For the outermost hollow body 4, said base 30 has a radius r′, where the length of said radius r′ is the same as the base radius of said cylinder which forms said outermost hollow body 4 and the radius of said base support 9;
b) Filling with at least one active agent the innermost internal volume of said hollow bodies, called core 5;
c) Closing said innermost hollow body 2 with a lid which is the top end 14 of said cylinder which forms said innermost hollow body 2, where said top end 14 has a radius the same as the base radius of said cylinder which forms said innermost hollow body 2;
d) Filling the hollow body 3 with at least one active agent;
e) Inserting inside said hollow body 3 said innermost hollow body 2, where the top end 14 of said innermost hollow body 2 rests on the bottom end 29 of said hollow body 3 and said base support 8 of said hollow body 2 forms the top end of said hollow body 3;
f) Filling the hollow body 4 with at least one active agent;
g) Inserting into said hollow body 4 said hollow body 3 inside which said innermost hollow body 2 is inserted, where the base support 9 of said hollow body 3 forms the top end of said hollow body 4;
h) Repeating said steps f and g for each further hollow body included in said device.

In one embodiment, said bottom ends 28, 29, 39 and said base supports 8, 9 are integral with said side walls 11, 12, 13, respectively.

In a further embodiment, said bottom ends 28, 29, 39 and said base supports 8, 9 are separate from said side walls 11, 12, 13, respectively.

In a preferred embodiment, said at least one bottom end 28, 29, 30 and said base supports 8, 9 have at least one through-hole, where said at least one through-hole opens out inside said empty volume, i.e. core or annular space 6, 7. Said at least one through-hole is intended for filling of said hollow body, for so-called debubbling. Once filling has been performed, said through-hole is suitably sealed, for example with a glue as described below, or with a droplet of the biodegradable material itself.

The method according to the present invention allows advantageously distribution of the at least one active agent inside said volumes 5, 6, 7 without empty spaces remaining inside them. Preferably, said volumes inside the aforementioned passages are filled with said at least one active agent until they are completely filled. The insertion of an inner hollow body 2, 3 inside the immediately more outer hollow body 3, 4 has the effect that the excess active agent is expelled, leaving volumes 6, 7 which are completely full.

Advantageously, the provision of a first hollow body which comprises a base support with an area greater than the base of the cylinder which forms the said hollow body and the same as the area of the base of the cylinder which forms the hollow body inside which said first hollow body is inserted facilitates alignment thereof, namely favours homogeneous distribution of the empty volume which is created between said outermost cylinder and said inserted cylinder.

The method described above does not impose any limitation on the overall number of layers and therefore may be repeated for any number of hollow bodies, and consequently volumes, which form the final device depending on the application and the clinical requirement.

The method does not impose any limitation on the volume of the single empty spaces and therefore it is possible to have configurations with volumes of drugs to be released which are the same and other configurations with different drug volumes, depending obviously on the clinical treatment.

The method according to the present invention, owing to the geometrical design of the system, advantageously allows the filling of hollow bodies inserted one inside the other with dimensions such that they may be injected and/or implanted, also for ophthalmic applications.

Said two hollow bodies inserted one inside the other are joined together by means of welding, gluing with surgical glues, or an interference-fit or friction-fit joint, using methods which are known to the person skilled in the art.

In a preferred embodiment, said welding method consists of laser microwelding which uses a very high concentration of energy, supplied in a very short periods of time, so as to cause rapid melting of the metal, keeping the thermal load to a minimum and creating small-size welding points which are clean, deep and of superior quality compared to those which can be obtained using conventional welding methods. In this embodiment, the welding may be performed by means of a standard very low power Nd-Yag laser providing a spot weld of 0.1-0.2 mm in an argon-based controlled inert atmosphere. Advantageously, this method ensures a high working speed, cleanliness, precision, a minimum thermal load, an optimum appearance of the weld, a very high mechanical strength, as well as the possibility of managing the penetration depth.

The surgical glues are chosen from the group which comprises surgical adhesives based on urethane, polyurethane glues, cyanoacrylate synthetic adhesives, for example 2-octyl cyanoacrylate and n-butyl cyanoacrylate, fibrin-based adhesives, gelatin and cross-linked gelatin obtained by synthesis, such as gelatin-resorcinol-formaldehyde, or with enzymatic crosslinking, such as mTG gelatin or photocrosslinked gelatin, albumin-based glue, dextran, chitosan, PEG or dual-component glues composed of a solution of purified bovine albumin serum and gluteraldehyde.

Merely by way of example, said interference-fit joint comprises connecting members such as splined profiles, where projections and recesses are formed in an axial direction along a section of said cylinder which forms the hollow body, and said base which is inserted inside said cylinder section also has projections and recesses in the axial direction.

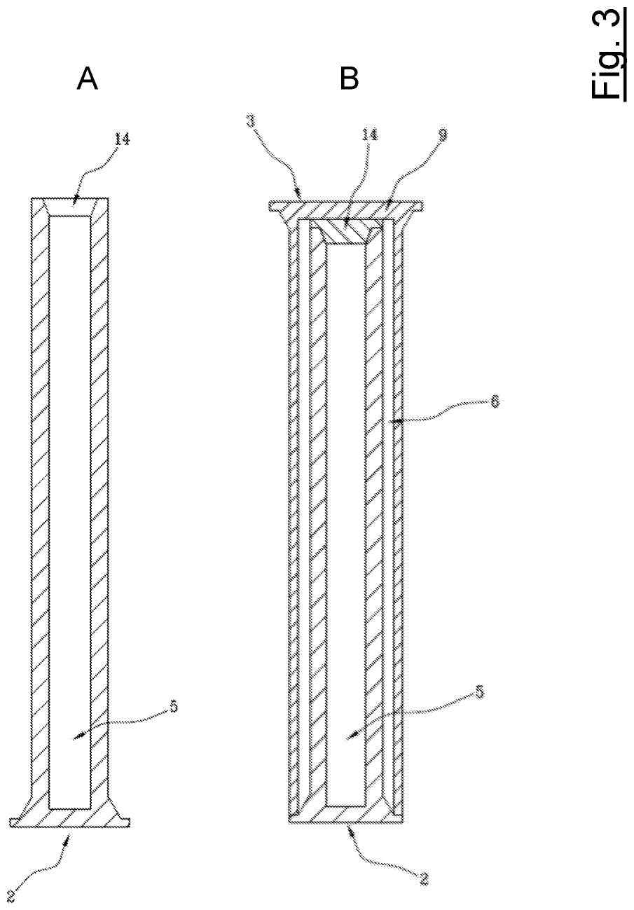

Merely by way of example, said friction-fit joint is a conical type joint, shown for example in FIG. 3, where a section of said cylinder 2 which forms the hollow body is tapered and said base 14, which is in turn tapered, is inserted in said tapered zone.

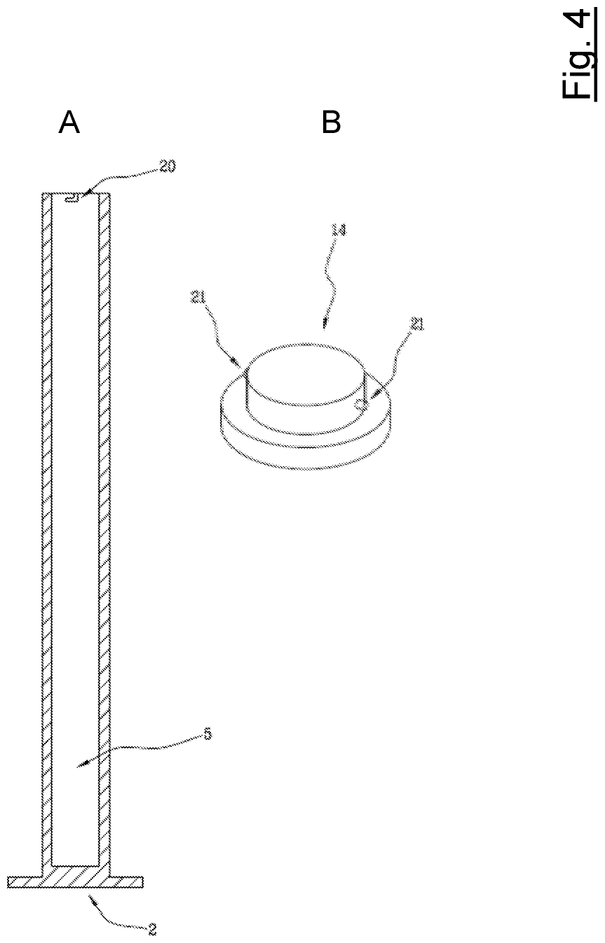

An example of an interference-fit joint is shown in FIG. 4. One end of said cylinder 2 which forms the hollow body has at least two L-shaped grooves 20 (view A) which cooperate with at least two projections 21 present on the lid 14 (view B). Said at least two projections cooperate with said grooves, locking the lid on the hollow body.

In a further embodiment, an implantable and/or injectable device as described above, filled with at least one active agent, is claimed.

Said at least one active agent, in one embodiment, is the same active agent contained in each of the empty volumes of the device. Alternatively, each of said empty volumes is filled with a different active agent. Alternatively, each of said empty volumes is filled with a mixture of at least two active agents.

Said active agent is in solid form, for example a powder or granules, or in the form of a liquid or gel.

Said at least one active agent is introduced as such, or else loose or dispersed in suitable dispersants or solvents which are known to the person skilled in the art.

The present invention also relates to an implantable and/or injectable device obtained according to the method described above.

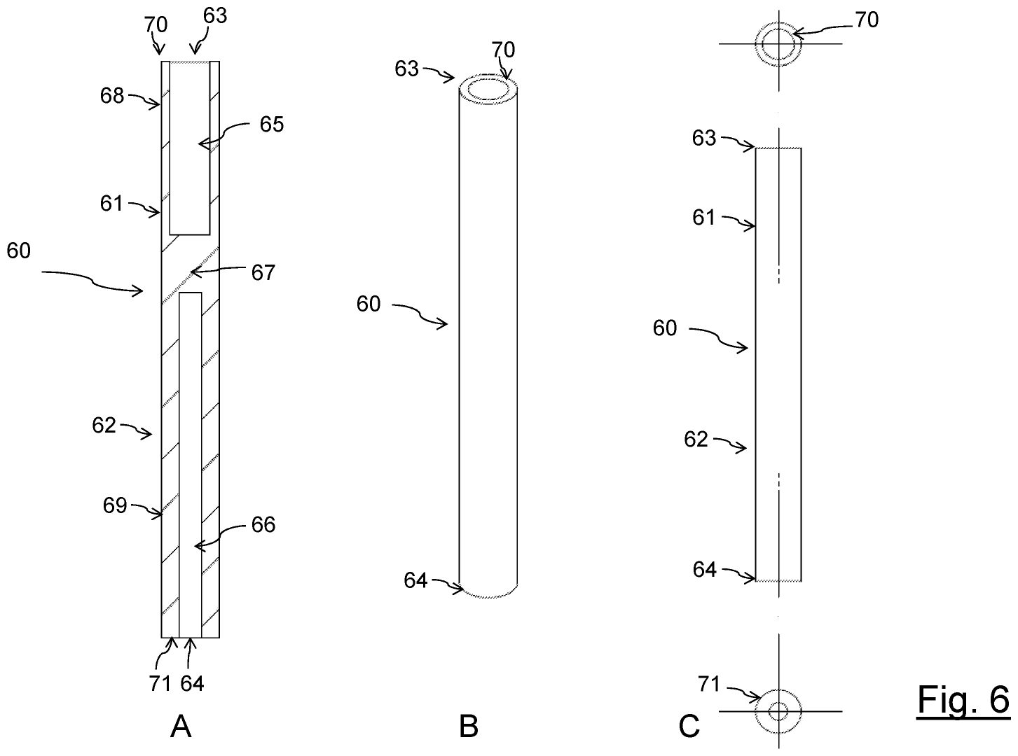

Furthermore the present invention relates to an implantable and/or injectable device which comprises two hollow volumes. Said device comprises a cylinder. With reference to FIG. 6, said device which comprises a cylinder 60 comprises a top portion 61 and a bottom portion 62. The top end 63 and bottom end 64 of said cylinder are open. From said openings on said top and bottom ends a first hollow volume 65 and a second hollow volume 66 are accessed respectively, whereby said first hollow volume 65 does not communicate with said second hollow volume 66.

Said first and second hollow volumes 65 and 66 are designed to house active doses.

Said first and second hollow volumes 65 and 66 are separated from each other by a partition 67 and are characterized in that the side wall 68 of said first hollow volume 65 has a thickness 70 which is smaller than the thickness 71 of the side wall 69 of said second hollow volume 66. Said partition 67 is impermeable to at least one active agent housed inside said first and second hollow volumes.

Said top end 63 and bottom end 64 are suitably closed with a base support (not shown) having a form and area practically the same as the form and area of the base of the cylinder 60.

Said side walls 68 and 69 and said base supports are impermeable continuous surfaces which become permeable depending on the post-implantation and injection time of said device. The different thickness of the side walls of said first and second hollow volumes is such that the time for dissolution of said side walls and therefore for outward opening of said first and second volumes is different, whereby said first hollow volume, which has side walls with a smaller thickness, opens outwardly first compared to said second hollow volume, which has side walls of greater thickness.

In one embodiment, said first and second hollow volumes have the same volume. In a further embodiment, said first and second hollow volumes have different volumes.

Said device consists of a biodegradable material which is preferably magnesium or a magnesium alloy. Magnesium alloys such as aluminium, lithium, calcium, zinc or manganese alloys constitute particularly preferred embodiments.

In a preferred embodiment, said device is made of magnesium alloy JDBM, an alloy of Mg-2.5Nd-0.2Zn-0.4Zr (wt %, JDBM) which has good mechanical and corrosion-resistance properties. Alternatively, said alloy is JBDM-2, Mg-2.2Nd-0.1Zn-0.4Zr (wt %, indicated as JDMB-2).

In one embodiment, where said implantable and/or injectable device is for ophthalmic applications, said device is a cylinder and has a height of about 10 mm, or about 5 mm and a diameter of the circular base of about 0.8 mm, 0.7 mm, 0.6 mm or 0.5 mm. Where said device is an injectable device and a standard needle is used for injection, said diameter of the circular base is 0.5 mm. For example, the thickness 70 of the side wall 68 of said first hollow volume 65 is equal to about 0.075 mm and the thickness 71 of the side wall 69 of said second hollow volume 66 is equal to about 0.150 mm. In one embodiment, said first hollow volume has a height of about 1.5 mm and said second hollow volume has a height of about 3 mm. In this embodiment, said first hollow volume and said second hollow volume have a volume which is practically the same.

The present invention relates furthermore to a method for manufacturing an implantable and/or injectable device according to this embodiment for the controlled release of a quantity of active agent.

Said method comprises:
providing a cylinder made of biodegradable material;
drilling said cylinder at the top end and bottom end, using drill bits with a different diameter on said top end and said bottom end, operating in such a way as to leave a partition separating a first hollow volume obtained by drilling said top end and a second hollow volume obtained by drilling said bottom end;
filling said first and second hollow volumes with at least one active agent;
closing said first hollow volume and said second hollow volume with a base support.

In one embodiment, said drilling is a microdrilling operation of the mechanical type, carried out in a manner similar to conventional mechanical microdrilling, but using drill bits with an extremely small diameter (<1 mm). Said microdrilling is performed using extremely precise and stable machinery which limit the run-out of the rotating bits and are equipped with high-performance spindles which ensure high speeds of rotation such as to compensate for the very small dimensions of the tools and ensure an adequate peripheral cutting speed.

In a further embodiment, said microdrilling is performed by means of electrical discharge machining or plunge spark machining.

The present invention also relates to the programmed-release implantable/injectable device according to the present invention for use in the treatment of pathologies which require the administration of at least one active agent which is repeated and programmed over time, for example every 2, or every 3 or every 4 weeks, or every 6, every 8, every 10 or every 12 weeks.

In a preferred embodiment, said pathologies are ocular pathologies and said device can be injected into the posterior eye chamber. By way of example, said ocular pathologies are chosen from among: exudative age-related macular degeneration, diabetic macular oedema, diabetic retinopathy, macular oedema from retinal venous occlusion, myopic macular degeneration and said at least one active agent is an anti-VEGF drug.

In a further embodiment, said pathologies are ocular pathologies which require treatment with active agents different from anti-VEGF drugs, for example macular oedema, where said at least one active agent is cortisone and/or at least one NSAID; inflammatory macular oedema, where said at least one active agent is cortisone and/or at least one immunosuppressant.

In one embodiment, said ocular pathology is an atrophic (or dry) age-related macular degeneration and said at least one active agent is chosen from the group which comprises: Anti-complement Inhibitors such as for example Pot-4, JPE1375, ARC1905, APL-2, Zimura, Eculizumab, immunomodulators such as glatiramer acetate, antioxidants such as OT-551, Fenretinide and/or Ciliary Neurotrophic Factor, Brimonidine, Doxycycline.

For example, said at least one active agent is chosen from the group comprising: adPEDF.11, AGN211745, Zybrestat, Sirolimus, ATG003, Bevacizumab, Ranibizumab, Pegaptanib, Aflibercept, Brolocizumab, Faricimab, Combercept, Abicipar, VEGF Trap, Vatalanib, Pazopanib, TG101095/TG100801, AL-39324, AG013958, JSM6427, PF-04523655 (REDD14NP), Ciliary Neurotrophic Factor, Fenretinide, OT-551, POT-4, Glatiramer Acetate, Anti-FGF2, tyrosine kinase inhibitors such as Sunitinib, anti-angiopoietin-2 such as RG7716, antibodies such as anti-endoglin, and tissue factor target protein such as ICON-1.

In a further embodiment, said pathology is hypercholesterolemia and said at least one active agent is Evolocumab, on its own or in combination with other hypolipidemic drugs. The device according to the present invention is of specific interest for use in treatment of homozygous familial hypercholesterolemia. In said pathology, which is a rare hereditary disease in which the LDL cholesterol levels are higher than normal from birth, it is required to administer Evolocumab subcutaneous at least once, preferably twice a month. The device according to the present invention, implanted subcutaneously, advantageously solves the problem of repeated injections.

In a further embodiment, said pathology is arterial hypertension. The device according to the present invention, implanted subcutaneously, is filled with one or more active agents chosen from the group which comprises ACE inhibitors, angiotensin II receptor antagonists, calcium antagonists, diuretics, alpha blockers, beta blockers, alpha-beta blockers, centrally acting sympatholytic drugs, renin-angiotensin-aldosterone system inhibitors.

In a further embodiment, said device is used for post-surgical prevention. For example, in open surgery or laparoscopy, the device is arranged in position so as to release in a programmed manner active agents such as anti-inflammatory drugs, cortisone-based drugs, antibiotics, antimetabolites or anticancer drugs.

In a further embodiment, said device is intended for use in the treatment and/or prevention of migraine and said device is filled with a monoclonal antibody which is an inhibitor of the calcitonin gene-related peptide (Cgrp) receptor, for example Erenumab.

In a further embodiment, the use of the programmed-release device as a contraceptive is claimed here. In this embodiment, the device is implanted within the vagina and/or subcutaneously and, in addition to use as a contraceptive, is used for the treatment of gynaecological and/hormonal disturbances.

1. An implantable and/or injectable device made of biodegradable material which comprises two hollow volumes where said device comprises a cylinder, which comprises a top portion and a bottom portion, where the top end and bottom end of said cylinder are open and via said top and bottom openings a first hollow volume and a second hollow volume are accessed respectively, where said first hollow volume and second hollow volume are separated from each other by a partition and are characterized in that the side wall of said first hollow volume has a thickness which is smaller than the thickness of the side wall of said second hollow volume.

2. The device according to claim 1, wherein said top end and bottom end are closed by a base support having a form and area practically the same as the form and the area of the base of the cylinder.

3. An implantable and/or injectable device which comprises at least two hollow bodies inserted one inside the other, where the walls of said at least two hollow bodies are made of biodegradable material, where said at least two hollow bodies inserted one inside the other delimit a volume, called core, inside the innermost of said hollow bodies and one or more volumes, called annular space, situated between said innermost hollow body and the hollow body on the outside thereof, characterized in that said hollow bodies are cylinders and each of said hollow bodies which form said implantable and/or injectable device, except for the outermost hollow body, characteristically consists of a cylinder which has a bottom end and a base support, wherein said base support has an area A and said base support has an area; said areas of said base supports being the same as or greater than the areas of the base of the cylinder inside which said hollow body will be inserted, and being able to be inserted inside any further hollow body inside which said hollow body will be inserted.

4. The device according to claim 2, wherein said base supports have a circular shape.

5. The device according to claim 3, wherein said hollow bodies are cylinders with a circular base.

6. The device according to claim 4, wherein the lengths of said radii of said base supports are the same as the lengths of the base radius of the cylinder inside which said hollow body will be inserted.

7. The device according to claim 1, wherein said hollow bodies and said cylinder made of biodegradable material are made of magnesium or a magnesium alloy.

8. The device according to claim 1, wherein said device has a height of about 10 mm, or about 5 mm and a diameter of the base, where circular, or of the circle inside which said base can be inscribed, equal to about 0.8 mm, or 0.7 mm, 0.6 mm or 0.5 mm or has a height of up to 40 mm and a diameter of the base, where circular, or of the circle inside which said base can be inscribed, of up to 4 mm.

9. The device according to claim 1, wherein the side walls of each of said cylinders and, where present, said partition which separates said first and said second hollow volumes, are an impermeable continuous surface and, preferably, at least one or both the bases of said cylinders are also impermeable continuous surfaces which become permeable depending on the post-implantation and/or injection time of said device.

10. The device according to claim 1, wherein said first and second hollow volumes or said core and said at least one annular space are filled with one or more active agents.

11. A method for manufacturing, filling and closing an implantable and/or injectable device for the controlled release of an active agent, wherein said method comprises the following steps:
a) providing single open hollow bodies, each hollow body being a cylinder and each comprising side walls and the bases, each of said open hollow bodies which will form the inserted hollow bodies having on said bases a base support with an area, where the areas of said base supports are the same as or greater than the areas of the base of the cylinder inside which said hollow body will be inserted, respectively smaller than the area of the base of the further cylinder inside which said hollow body will be inserted;
b) filling with at least one active agent the innermost internal volume of said hollow bodies, called core;
c) closing said innermost hollow body with a lid which is the top end of said cylinder which forms said innermost hollow body;
d) filling the hollow body with at least one active agent;
e) inserting inside said hollow body said innermost hollow body, where the top end of the said innermost hollow body rests on the base of said hollow body and said base support of said hollow body forms the top end of said hollow body;
f) filling the hollow body with at least one active agent;
g) inserting into said hollow body said hollow body inside which said innermost hollow body is inserted, whereby the base support of said hollow body forms the top end of said hollow body;
h) repeating said steps f and g for each further hollow body included in said device.

12. The method according to claim 11, wherein said base and said base support are integral with said side walls, respectively, or form a separate element.

13. The method according to claim 11, wherein said two hollow bodies inserted one inside the other are joined together and/or said bottom end is joined to said side wall by means of welding, gluing with surgical glues, an interference-fit joint, or a friction-fit joint.

14. The method according to claim 11, wherein said active agent is in solid form, for example a powder or granules, or in the form of a liquid or gel.

15. A method for manufacturing, filling and closing an implantable and/or injectable device for the controlled release of an active agent, wherein said method comprises the following steps:
a) providing a cylinder made of biodegradable material;
b) drilling said cylinder at the top end and bottom end, using drill bits with a different diameter on said top end and on said bottom end, operating in such a way as to leave a partition separating a first hollow volume obtained by drilling said top end and a second hollow volume obtained by drilling said bottom end;
c) filling said first and second hollow volumes with at least one active agent;
d) closing said first hollow volume and said second hollow volume with a base support.

16. An implantable/injectable device for the controlled release of quantified amount according to claim 10 for the use in the treatment and/or prevention of pathologies which require the administration of at least one active agent repeated and programmed over time, preferably every 2, or every 3 or every 4 weeks, or every 8, or every 10 or every 12 weeks.

17. The device for use according to claim 16, wherein said pathologies are chosen from the group which comprises: ocular pathologies, for example exudative age-related macular degeneration, diabetic macular oedema, diabetic retinopathy, macular oedema from retinal venous occlusion, hypercholesterolemia, preferably homozygous familial hypercholesterolemia, arterial hypertension, migraine, gynaecological and/hormonal disturbances.

18. The use of the device according to claim 10 as a contraceptive.

19. The device according to claim 10, for use as a contraceptive.
